(19) United States
(12) Patent Application Publication
(10) Pub. No.: US 2026/0334778 A1
(43) Pub. Date: Oct. 29, 2026

(54) OPTICAL COHERENCE TOMOGRAPHY WITH DIGITAL DOWN CONVERSION

(71) Applicant: Carl Zeiss Meditec, Inc., Dublin, CA (US)

(72) Inventor: Matthew Muller, Livermore, CA (US)

(73) Assignee: Carl Zeiss Meditec, Inc., Dublin, CA (US)

(21) Appl. No.: 19/661,235

(22) Filed: Apr. 28, 2026

Related U.S. Application Data
(60) Provisional application No. 63/796,857, filed on Apr. 29, 2025.

(51) Int. Cl. A61B 3/10; G01B 9/02; G01B 9/02055; G01B 9/02091

(52) U.S. Cl. CPC A61B 3/102; G01B 9/02044; G01B 9/02069; G01B 9/02072; G01B 9/02083; G01B 9/02091

(57) ABSTRACT

An optical coherence tomography (OCT) system/method/device that implements two sequential frequency down conversions to reduce electronic processing requirements. The system identifies and processes only the part of an OCT scan that is relevant for clinical use.

### CROSS-REFERENCE TO RELATED APPLICATIONS

This application claims priority to, and the benefit of, Provisional Patent Application No. 63/796,857, filed Apr. 29, 2025, and titled “Swept Source Optical Coherence Tomography with Digital Down Conversion,” which is incorporated by reference herein in its entirety for all purposes.

### FIELD

The present disclosure is generally directed to high speed, lower cost optical coherence tomography (OCT) system.

### BACKGROUND

As OCT A-scan speeds increase, proportionally more data is generated, which drives the cost of the data acquisition and collection (e.g., the signal detector and signal digitizer), the data processing and analysis (e.g., GPU and/or cloud computing resources), and the data storage (locally on a personal computer (PC) and/or on the cloud). This leads to higher speed and higher capacity components, which increase the cost of the overall OCT unit. For example, OCT data is typically collected, processed and stored using a fixed depth (axial scan) range, typically 2-3 mm for SD-OCT and 6 mm for SS-OCT. The full-depth range data is sent to a PC for processing, analysis and storage. This results in higher component and/or cloud computing and storage costs, as well as higher transfer and processing time requirements. One example that attempts to address some of these problems is found in U.S. Pat. No. 10,488,178, which describes the use of frequency down conversion in an OCT system.

In various embodiments, the present disclosure reduces the cost of OCT components without reducing its effective speed.

In various embodiments, the present disclosure reduces the time required for data transfer and processing, and thus improve user experience.

### SUMMARY

The benefits are achieved in a system/method for reduced performance requirements of individual component(s) within an optical coherence tomography (OCT) system while maintaining a high overall performance of the OCT system.

To achieve this, the present disclosure provides several improvements to reduce the required data processing rate and data transfer rate between an acquired (analog) OCT signal acquisition and the data processing, electronic components, at least some of which may be digital circuit components. A first step towards keeping a high OCT signal acquisition rate while reducing the data processing rate requirements of the circuit components, the frequency band of the acquired OCT signal is down converted to a lower frequency band, which reduces the data processing speed requirements of the electronic components.

Additionally, only a select portion (e.g., target portion or signal window) of an acquired OCT signal (scan) is captured and transferred to the electronic components for processing. This further reduces the speed requirements of communication busses and data processing rate requirements of electronic components. The signal window is chosen to correspond to the part of the acquired OCT signal (e.g., on an A-scan by A-scan basis) that correspond to clinically relevant data. For example, if taking an OCT scan of the retina of an eye, only the portion of an A-scan that corresponds to the retina (e.g., tissue region of region, ROI) is selected for transfer to, and processing by, electronic components. As is known in the art, an eye (and its retina) is curved, and the radius of this curvature is much larger than the retina thickness (e.g., the tissue ROI). To further reduce the amount of OCT signal information that is transferred and processed, the size (e.g., along the axial dimension of an A-scan) of signal window of an A-scan is chosen to correspond to a predefine axial span expected to correspond to the tissue ROI. This signal window is shifted up and down at different A-scans to follow the curvature of the eye, so each select signal window of data of an A-scan includes additional (header/meta) position-information specifying the position of the shifted signal window within the original A-scan. Thus, once the OCT data has been processed, and an image is to be constructed, the position-information is used to position the processed signal window within its original A-scan position and thereby reconstruct the original curvature of the eye. A solution for how to determine the position of the signal window within each newly acquired A-scan without undue burden on the processing/component cost of the OCT system is also provided.

A method of reducing the amount of raw data acquired from an OCT system, may include: digitizing an OCT signal from the OCT system; performing a first down conversion and decimation on the digitized OCT signal to isolate a group of OCT signal frequencies of interest within a first low frequency bandwidth; finding a bandwidth and a center frequency that characterizes the group of OCT signal frequencies of interest within the first low frequency bandwidth; and using the bandwidth and center frequency to perform a second down conversion and decimation step to further isolate the group of OCT signal frequencies of interest within a second low frequency bandwidth that is smaller than the first low frequency bandwidth.

Alternatively, the method of reducing the amount of raw data acquired from an OCT system, may include: performing a first down conversion on an OCT signal from the OCT system to isolate a group of OCT signal frequencies of interest within a first low frequency bandwidth; filtering and digitizing the first low frequency bandwidth of the OCT signal; finding a bandwidth and a center frequency that characterizes the group of OCT signal frequencies of interest within the first low frequency bandwidth; and using the bandwidth and center frequency to perform a second down conversion and decimation step to further isolate the group of OCT signal frequencies of interest within a second low frequency bandwidth that is smaller than the first low frequency bandwidth.

An optical coherence tomography system may further include: a light source for generating a beam of light; a beam divider for directing a first portion of the light into a reference arm and a second portion of the light into a sample arm; optics for directing the light in the sample arm to one or more locations on a sample; a detector for receiving light returning from the sample and reference arms and generating an analog OCT signal in response thereto; a processor implementing the following steps: digitizing the OCT signal; performing a first down conversion and decimation on the digitized OCT signal to isolate a group of OCT signal frequencies of interest within a first low frequency bandwidth; identifying a bandwidth and a center frequency that characterizes the group of OCT signal frequencies of interest within the first low frequency bandwidth; and using the bandwidth and center frequency to perform a second down conversion and decimation step to further isolate the group of OCT signal frequencies of interest within a second low frequency bandwidth that is smaller than the first low frequency bandwidth.

In the first down conversion and decimation step of this system, the digitized OCT signal is separately mixed with a plurality of local oscillators to produce a plurality of intermitted heterodyned signals, and a select intermittent heterodyned signal is identified as the bandwidth that characterizes the group of OCT signal frequencies of interest.

In this case, the select intermittent heterodyned signal is of highest signal strength among the plurality of heterodyned signals.

The system may be a swept source OCT system, where the OCT signal is resampled using a clock that is derived from the wavenumber k dependence of a sweep; and a phase correction term is applied to the heterodyned signal based on a local oscillator used in the first down conversion. The phase correction term may be obtained by resampling the signal phase of the local oscillator with the same time values used to resample the OCT signal. Alternatively, the phase correction term may be modeled using an estimate of the local oscillator frequency, and an error in this estimation may be identified as an increase in a point-spread function (PSF) width, and adjust accordingly.

Other objects and attainments together with a fuller understanding of the disclosure will become apparent and appreciated by referring to the following description and claims taken in conjunction with the accompanying drawings.

Several publications may be cited or referred to herein to facilitate the understanding of the present disclosure. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1 is a block diagram indicating the signal paths through a digitizer, in accordance with various embodiments;

FIG. 2 provides block diagram of an ADC (AD9699) for reference, in accordance with various embodiments;

FIGS. 3A and 3B illustrate the digital down conversion process in the frequency domain, in accordance with various embodiments;

FIG. 4 illustrates a method for determining when to switch between NCOs to ensure that the part of an OCT signal (A-scan) containing the target tissue (e.g., the retina) is continuously selected from A-scan to A-scan, in accordance with various embodiments;

FIG. 5 illustrates the effect that digital down conversion decimation has on the data size, in accordance with various embodiments;

FIG. 6 shows the reassembly of row-shifted A-scan 33 of FIG. 5, in accordance with various embodiments;

FIG. 7 shows the advantage of a secondary decimation stage, in accordance with various embodiments;

FIG. 8 shows an example of using analog components instead of digitally using an FPGA, as described above, and in accordance with various embodiments;

FIG. 9 is a related analog embodiment. Tracking can be implemented by adapting the 4-digital filter embodiment above with analog down conversion and analog filtering and peak detection, in accordance with various embodiments;

FIG. 10 illustrates a generalized frequency domain optical coherence tomography system used to collect 3D image data of the eye suitable for use with the present system, and in accordance with various embodiments;

FIG. 11 shows an exemplary OCT B-scan image of a normal retina of a human eye, and illustratively identifies various canonical retinal layers and boundaries, in accordance with various embodiments;

FIG. 12 shows an example of an en face vasculature image, in accordance with various embodiments;

FIG. 13 shows an exemplary B-scan of a vasculature (OCTA) image, in accordance with various embodiments; and

FIG. 14 illustrates an example of a computer system (or computing device or computer), in accordance with various embodiments.

### DETAILED DESCRIPTION

One of the most expensive components in an OCT system, and particularly in a swept source OCT (SSOCT) system, is the digitizer circuit, which converts an acquired (analogy) OCT scan signal to a digital signal representation. This digitizer circuit has a very high sample rate (especially for SSOCT) to enable capture of the signals from reflectances that are near the maximum path length difference (see below for a more detail discussion of OCT systems). In addition, the rest of the system processes this high frequency data in real time during the scan signal acquisition. An approach for reducing the high frequency data requirements is discussed below.

Herein is presented an alternative OCT system design that would use a fast detection circuit followed by downsampling the data to an intermediate frequency, e.g., by using a numerically controlled oscillator (NCO) to heterodyne the OCT signal to reduce the bandwidth, followed by a digital downsampling step. Generally, heterodyne may be understood as frequency down-conversion, where high-frequency signals are shifted to a lower, more a manageable frequency band. For example, Heterodyne relates to the production of a lower frequency from the combination of two (e.g., optionally similar) high frequencies, such as used in radio transmission.

A discussion of the effect of heterodyning on the reconstruction of SSOCT data is first provided. Specifically, if the sweep is nonlinear in optical wavenumber vs time, the signal is resampled. Generally, a wavenumber may be understood as the number of wavelengths per unit distance. This resampling complicates the effects of mixing, resulting in phase errors in the output signal. An investigation of these phase errors led to the deriving of a phasor multiplier from the phase of the resampled local oscillator signal. By use of this phasor multiplier, the phase errors in the heterodyned signal may be corrected to produce a narrow point-spread function (PSF).

### Description of the Phenomenon

Heterodyning is the process of multiplying the input signal by a locally generated signal referred to as the local oscillator (LO) signal, which is approximately a single frequency. If, for simplicity, one considers the input signal to be a sine wave at frequency fᵢ, the product will be

$$= \frac{A B}{2} [ cos ( 2 π ( ( {f}_{i} - {f}_{L O} ) t ) ) - cos ( 2 π ( ( {f}_{i} + {f}_{L O} ) t ) ) ] \qquad ( 1 )$$

producing signals at sum and difference frequencies of the two components. On is typically only interested in the difference frequency product because that is at a lower frequency and easier to process. The sum frequency component is usually filtered out.

In SSOCT, a single reflectance in the sample arm will produce a signal that is nearly a pure sine wave. The reconstruction of this signal is done via a Fourier transform, producing an output that is approximately a delta function, neglecting considerations of windowing, finite signal length, etc.

The signal from a thin reflector is at a single frequency when the signal is linear in wavenumber k. Since the signal is acquired via sampling linearly in time, it is required that the wavenumber k be linear in time as well. This condition is generally not met. To fix this, one may resample the signal using a clock that is derived from the k dependence of the sweep.

Typically, when this resampling of the raw SSOCT data is done, one gets a signal that is suitable for reconstruction using the Fourier transform. However, the heterodyned signal is complicated by the fact that the input has been mixed with the LO signal. When the product is resampled, this has the effect of spreading the spectrum of the LO signal so it is no longer a single frequency. Since the phase of the difference frequency output is the difference between the phases of the two input signals, a phase error has been introduced that causes an increase in the width of the PSF.

### Phase Correction

If one adds a time-dependent phase error φ_{LO}(t) to the LO signal and keeps only the difference frequency component, the heterodyned (mixed) signal Sₕ(t) of equation (1) becomes

$${S}_{h} ( t ) = \frac{A B}{2} cos ( 2 π ( ( {f}_{i} - {f}_{L O} ) t - {φ}_{L O} ( t ) ) ) \qquad ( 2 )$$

As it would be understood, AB/2 represents an arbitrary amplitude related to the two signals being mixed, fᵢ represents the OCT signal, f_{LO} is the local oscillator, and t is time. The phase error is removed from this expression before reconstruction can be performed. This can be done by obtaining the analytic signal corresponding to Sₕ and multiplying by a complex phasor Φ given by

Φ=exp(i·φ_{LO}(t))

S_{h,corrected}=Re(SAₕ(t)*Φ) (3)

where SA is the analytic signal corresponding to S, i identifies the OCT signal being processed (e.g., a specific A-scan within a B-scan), and Re limit the solution to the real values.

As an aside and as is known in the art of mathematics and signal processing, an analytic signal is a complex-valued function that has no negative frequency components. The real and imaginary parts of an analytic signal are real-valued functions related to each other by the Hilbert transform.

The phase correction term may be obtained by resampling the LO signal phase with the same time values used to resample the OCT signal. If the actual LO signal phase is not available, it may be modeled using an estimate of the LO frequency. If there is an error in this estimate, one will see an increase in the PSF width, and adjust accordingly.

Return to general discussion.

OCT B-scans of an eye retina contain only a small portion of localized depth information (e.g., signal window) over a relatively large depth (axial scan) range. In most cases, the ratio of useful to non-useful information is 1:3 to 1:6, which presents an opportunity to filter and transmit only the useful data to a computing device (e.g., electronic components) as a means to realize cost and time savings.

In the present embodiment, an OCT signal (e.g., acquired, analogy OCT scan signal) is detected by a balanced detector and sent to a digitizer that:
1) Digitizes the analog signal using an analog-to-digital converter.
2) Down converts the digitized signal by mixing it with a numerically controlled oscillator (NCO). The NCO frequency is set to down convert the useful frequency band (e.g., the frequency band of the OCT signal that contains useful information, e.g., information pertaining to the retina or other target tissue) to a lower frequency.
3) Low-pass filters the down converted signal to remove unwanted information outside of the useful frequency band.
4) Decimates the data to reduce its size while maintaining all of the information in the useful frequency band (e.g., the useful frequency band includes information of the target object being scanned, e.g., the retina).
5) Sends the decimated data to an FPGA (field programmable gate array, or other type of configurable hardware component, such as Programmable Logic Device (PLD), Complex Programmable Logic Devices (CPLD), or Simple Programmable Logic Devices (SPLD)), which performs additional filtering and (optional) decimation on the data to identify and optimize the size of the data to send to a PC (e.g., computing device/electronic circuits/components). The FPGA also performs filtering to track the position of the retina across the B-scan.
6) The FPGA organizes and packetizes the data to send to the PC, and typically sent to a memory space shared with the GPU (graphics processing unit) or GPGPU (general-purpose computing on graphics processing units) or CPU (central processing unit) for reconstruction. The FPGA also adds the NCO “channel” and decimation factor used to down convert each A-scan to its header so that the reconstructed B-scan can be properly displayed. As discussed below, a preferred embodiment uses multiple NCO's, each providing a different LO frequency and designated as a different NCO channel. Each NCO selects shifts (up or down) a signal window by a different axial amount within an acquired A-scan.

Typically, one would change an NCO frequency by writing to a register, but this is relatively slow. With this typical approach, it would not be feasible to update the NCO frequency during a B-scan since one would not know exactly when (i.e. at which A-scan) the changed NCO frequency applied without some additional synchronization electronics. The present approach solves this problem by using NCO channels that can be rapidly changed during the time period between A-scans.

Unlike the prior art, the present approach stores the down conversion frequency (or the NCO channel) for each A-scan in the B-scan. This is necessary in order to apply depth offsets to the reconstructed B-scans and get a clinically usable image, as discussed below.

The present approach performs down conversion with a limited and finite number of frequency steps (NCO channels). The present NCO channels are derived from a highly stable sampling clock and provide reliable depth offsets for each A-scan. The present approach thus provides a simplified solution that avoids phase-lock loops or other complicated oscillation control systems. For example, the prior art would typically require a phase-locked loop to compensate for clock timing jitter and voltage-controlled oscillator drift, but the present approach avoids this problem.

Additionally, the present approach performs a second stage of filtering, down conversion, and decimation on the FPGA to optimize the data size that is sent to the GPU, GPGPU, or CPU for reconstruction. This is advantages because when down converting and decimating the data on an analog-to-digital converter (ADC or digitizer), the amount of decimation generally cannot be changed in real-time. Since the clinically useful width of the retina can vary considerably across a B-scan's field-of-view (FOV), particularly when certain retinal lesions, e.g. retinal detachments and macular edema, are present, the size of the depth window is limited to the maximum depth for clinical interpretation.

Optionally, down conversion and decimation could be performed in a single step using an FPGA placed after the ADC, but this may be sub-optimal since the full (undecimated) data set is then transferred to the FPGA, which drives up the cost of this component. The ADC performs a first stage of down conversion and decimation, with the decimation set so that the OCT signal covers the maximum depth for clinical interpretation across the patient population. This reduced data set is more efficiently sent to the FPGA, which further processes and decimates the data according to the size of each A-scan or of the maximum depth in a particular B-scan.

An advantage of the present two-stage down conversion and decimation is that the data sent to the GPU, GPGPU, or CPU for a subject with a generally uniform retinal thickness (the typical case, even for many patients with retinal disease) will be lower than a single-stage implementation that accounts for atypical cases that have a much larger retinal thickness. For example, if the decimation is set to account for the 1% of B-scans that include very thick retinal features that are of clinical interest, the remaining 99% of the data will be larger than it needs to be.

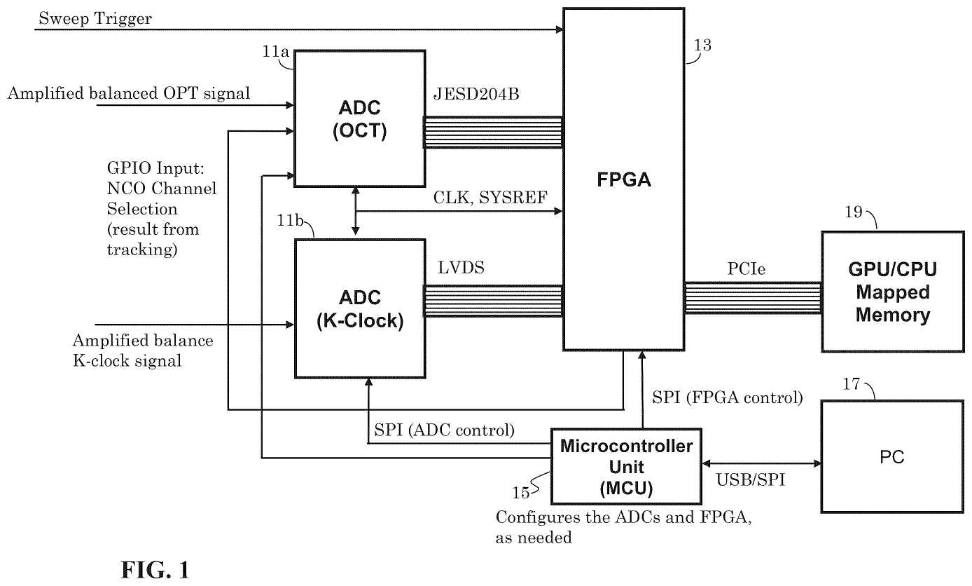

FIG. 1 is a block diagram indicating the signal paths through a digitizer, in accordance with various embodiments.

In this embodiment, an analog-to-digital converter (ADC) 11a/11b is used separately for the OCT and K-clock channels. This choice reduces the FPGA (13) cost by reducing the required serial transceiver speed (e.g., JESD204B, a JEDEC (Joint Electron Device Engineering Council) standard for a high-speed serial interface). Both ADCs 11a/11b and the FPGA 13 are configured via an SPI (Serial Data In) communication channel from the microcontroller 15, which is in turn controlled by the PC 17. The FPGA 13 provides the clock (CLK) and synchronization (SYSREF) signals to the ADCs 11a/11b to ensure that both the OCT and K-clock are well-aligned in time. The FPGA 13 receives a digitized, digitally down-converted, digitally filtered and decimated signal from the OCT ADC 11a over a JESD204B interface. The K-clock ADC 11b samples, preferably at a sampling rate that is an integer divisor of the OCT ADC 11a sampling rate, and sends the digitized data to the FPGA over a slower LVDS (Low Voltage Differential Signaling) interface. The FPGA 13 performs an additional digital filtering and optional decimation step on the OCT data sent from the OCT ADC 11a to optimize the data size for each A-scan or for each B-scan. The FPGA 13 stores the digitized data in a dual-port first-in-first-out (FIFO) buffer (not shown), and organizes and packetizes the data to be sent over PCIe (a high-speed bus communication standard) to GPU 19 mapped memory. Block memory on the FPGA 13 is used during this process to account for latency between the incoming and outgoing data.

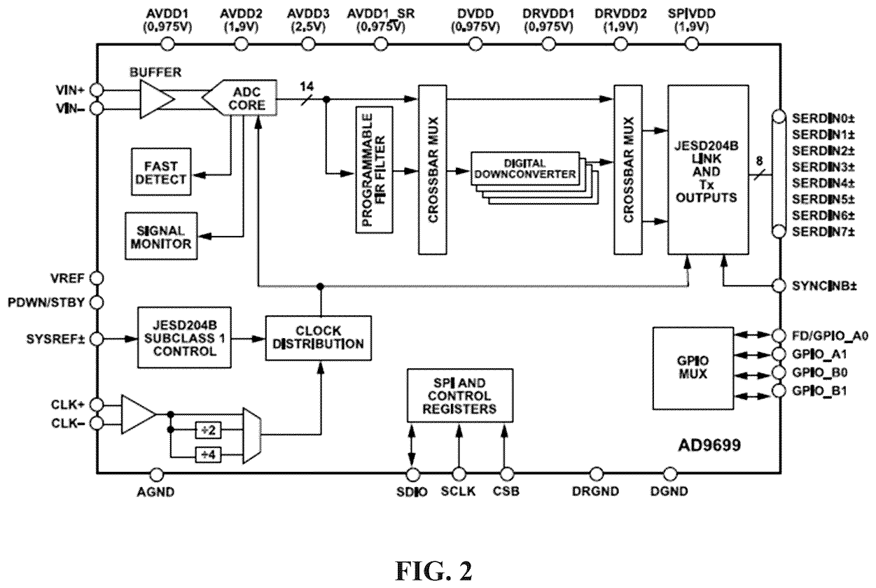

Digital down conversion is performed on the OCT ADC. An example of a suitable ADC is AD9699 from Analog Devices™ Corp. FIG. 2 provides block diagram of an ADC (AD9699) for reference.

With reference to FIGS. 1 and 2, the amplified balanced OCT signal (e.g., an acquired A-scan) is sent into the ADC at VIN+ and VIN−. After being digitized by the ADC core 21, it proceeds to one of four digital down converter modules 23. The down converted, filtered and decimated output is sent to the JESD204B serializer 25 and sent out to the FPGA 13. Registers 27 are set using the SPI interface (Serial Peripheral Interface), and the input clock and synchronization between devices, as well as selection of the NCO channel, is controlled via dedicated input pins for high timing accuracy and reliability. On the AD9699, changes to the NCO channel via the GPIO 29 (General Purpose Input/Output) require 8 sampling clock cycles, i.e. an NCO channel can change within 2.7 ns (nano seconds), which is considerably faster than the several hundred microseconds laser “off” time between A-scans (of a B-scan). It should be noted that on this and other commercially available ADCs, only the NCO channel can be rapidly switched; the remaining parameters associated with digital down conversion, including the filters and decimation values, are set via registers on the slower SPI interface with an unpredictable update time. It is therefore not possible with this ADC to change the amount of decimation during a B-scan to account for different A-scan retinal thicknesses. A different ADC may provide this capability.

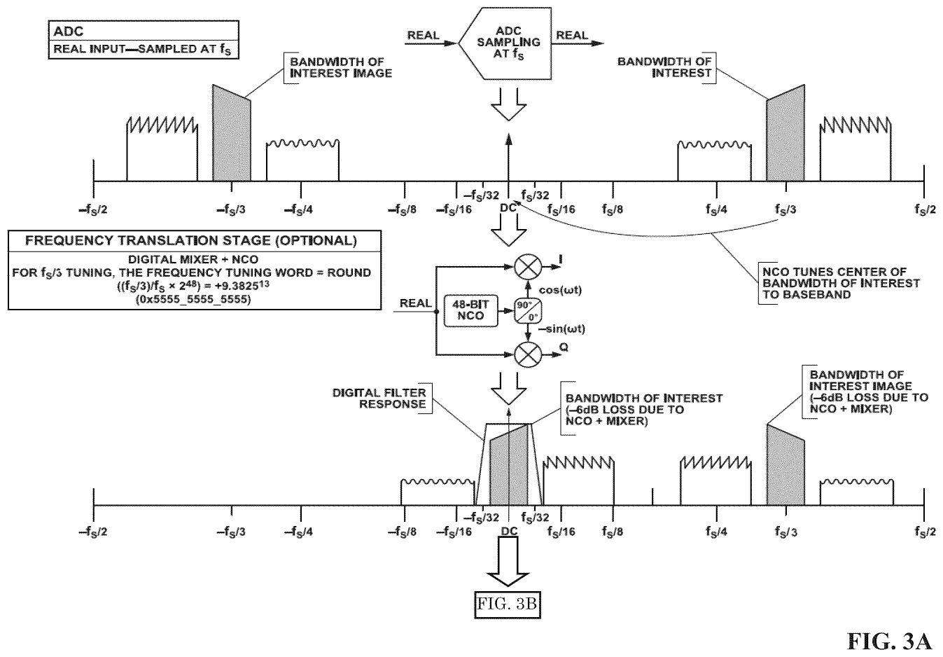

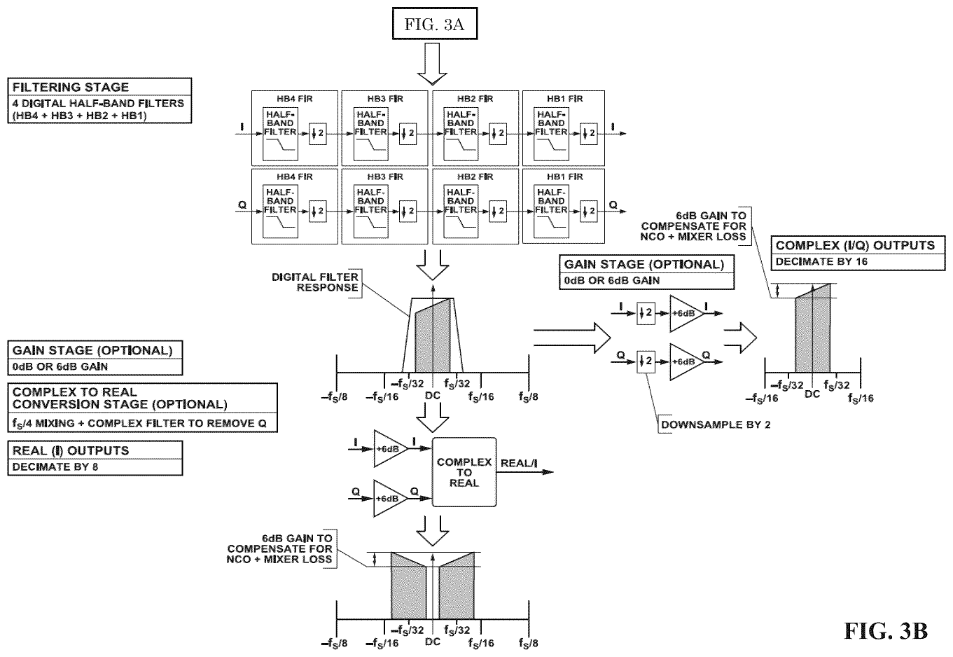

FIGS. 3A and 3B illustrate the digital down conversion process in the frequency domain. In this diagram, a “bandwidth of interest” corresponds to the region around the retina in an A-scan (approximately 1-2 mm in depth in tissue). The NCO frequency is selected so that it is mixed down to DC (as shown) for full-range OCT, or to a low frequency band so that quadrature demodulation is not required. The down converted bandwidth of interest is then filtered, optionally amplified, and decimated to remove the unneeded higher frequency data.

In this embodiment, the FPGA performs FIFO (first-in, first-out) data I/O, an additional digital filtering and decimation stage, and retinal finding and tracking. The frequency corresponding to the location of useful (retina, e.g., target tissue) data can be found by splitting the total digitized bandwidth into multiple sections of equal width. This is realized on the analog-to-digital converter (ADC) with separate NCO “channels”, each of which has a different NCO frequency, and a narrowband filter. The NCO channels can be rapidly switched between A-scans. In one embodiment, there are 16 NCO channels on the ADC and the total digitized bandwidth is split into 16 sections and scanned over 16 A-scans. In this embodiment, the signal strength of each NCO channel is calculated using the FPGA and thresholded to ensure a valid signal (retina) is present. The NCO channel with the maximum signal strength corresponds to the location of the retina.

Once the initial location of the retina is found, it can be tracked by further filtering the down converted and decimated retinal data on the FPGA. In one embodiment, the decimated bandwidth corresponding to the retinal data filtered with high and low pass filters, and the filtered signal strength is compared. In this embodiment, when the retina shifts axially, the filtered signal strength will also shift between the 4 sections.

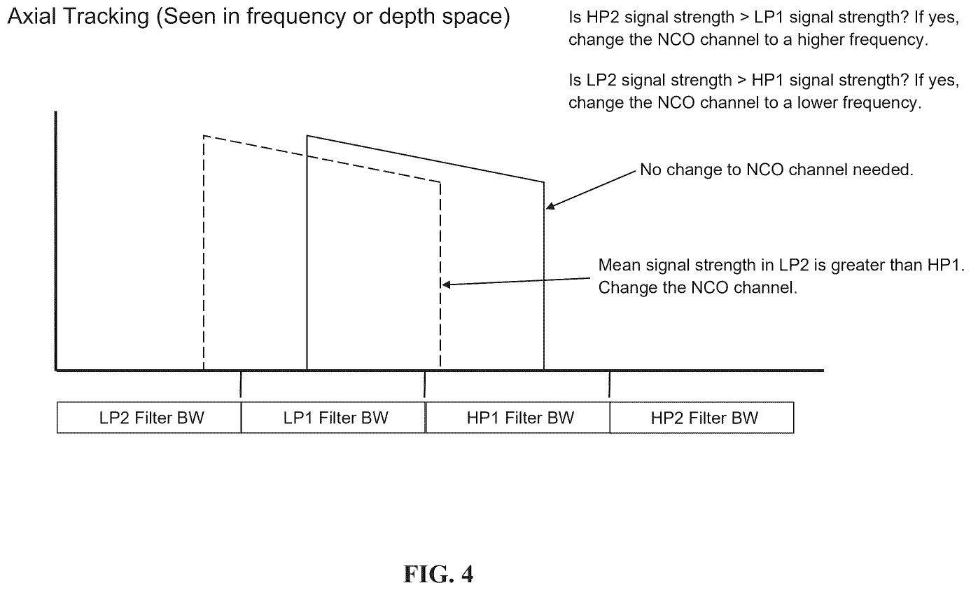

FIG. 4 illustrates a method for determining when to switch between NCOs to ensure that the part of an OCT signal (A-scan) containing the target tissue (e.g., the retina) is continuously selected from A-scan to A-scan. To ensure the NCO channel is switched only when the retina has moved “sufficiently far”, the signal strength in the outer sections are compared to the signal strength in the inner sections of the decimated bandwidth. In this embodiment, the filter bandwidths are preferably set to the NCO channel spacing. Although the decimated bandwidth appears to match the combined bandwidth of the four filters, this is not required. Those skilled in the art will recognize that, for this tracking implementation, the decimated data bandwidth is at least two times the selected filter bandwidth, and the bandwidth corresponding to useful (retina) data should be greater than one selected filter bandwidth. Either the decimated data from the ADC or from the secondary decimation stage on the FPGA can be used as input for axial tracking.

As stated previously, the decimated bandwidth sent from the ADC to the FPGA should correspond to the maximum retinal depth that is clinically desirable during a B-scan. The FPGA is configured to perform an additional digital filtering stage that serves to find the thresholded bandwidth of the useful (retina) data and to further down convert and decimate the data when desirable. The secondary stage down conversion and decimation settings are stored with each A-scan to enable reconstructing a clinically useful B-scan for visualization.

In one embodiment, the secondary stage down conversion and decimation is selected according to the maximum retinal bandwidth found across an entire B-scan. This is achieved by determining the maximum bandwidth of all A-scans in the B-scan and determining the appropriate decimation factor to avoid loss of clinically desired data. In this embodiment, the A-scans are filtered to determine their bandwidth, down converted and decimated, and finally sent to the GPU/CPU. In this embodiment, it may be required to read and write the A-scans to an external dual-port RAM memory module due to a limited amount of block memory available on the FPGA.

In a related embodiment, the secondary down conversion and decimation stage is performed during a spiral scan. In this embodiment, the scan is split up into decimation sections depending on the current retinal thickness, which typically decreases with radial distance from the macula. As the A-scans are captured, the bandwidth of useful retinal data is determined. When the retinal bandwidth decreases past a pre-determined value, the secondary stage down conversion and decimation is updated. In this embodiment, the A-scans are generally sorted according to decimation level, making the reconstruction processing in software more efficient.

In another related embodiment, the secondary down conversion and decimation stage is performed on each A-scan. The A-scans are stored in different memory locations according to their decimation and are stored with a laser sweep number so that they can be reassembled into a clinically useful image after reconstruction. When a certain number of A-scans of a certain decimation have been accumulated, they are sent as a block to the GPU/CPU for reconstruction.

The below figure illustrates the effect that digital down conversion decimation has on the data size. Note that the down conversion and decimation described on the ADC and FPGA occurs in the time domain. The effect of down conversion and decimation on the reconstructed B-scan is a shift in the depth position and a change in the depth window size, respectively, which is well-known to those familiar with the Fourier relationship between the signal received in a swept source OCT system and the reconstructed output.

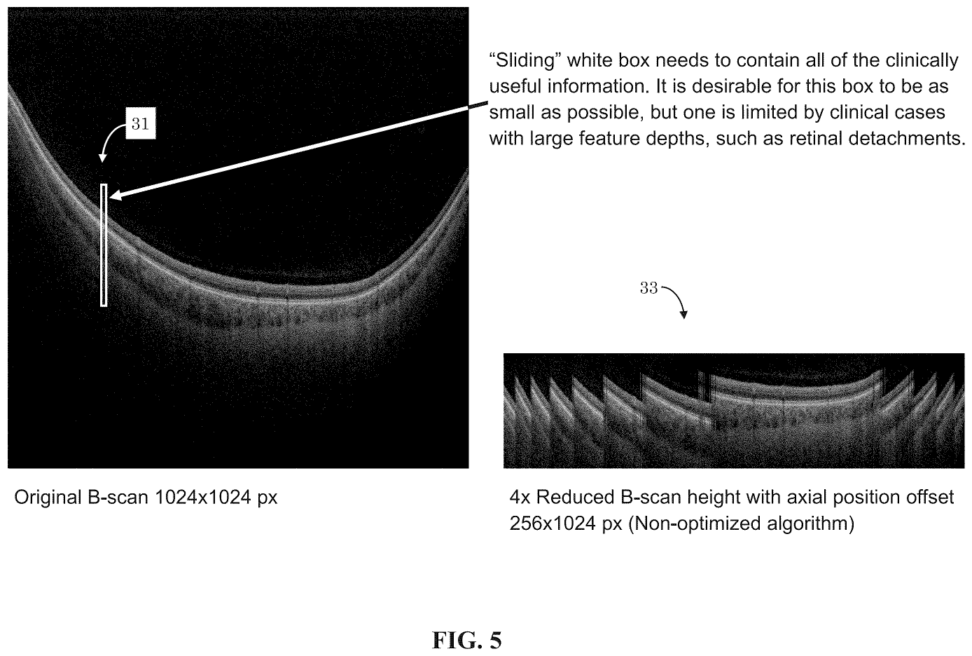

FIG. 5 illustrates the effect that digital down conversion decimation has on the data size. Note that the down conversion and decimation described on the ADC and FPGA occurs in the time domain. The effect of down conversion and decimation on the reconstructed B-scan is a shift in the depth position and a change in the depth window size, respectively, as would be understood by those familiar with the Fourier relationship between the signal received in a swept source OCT system and the reconstructed output.

With reference to FIG. 5, the sliding white box 31 is the depth window width that is determined by the decimation setting. The row-shifted A-scans 33 could be achieved using discrete NCO channels, each of which uses a different down conversion frequency.

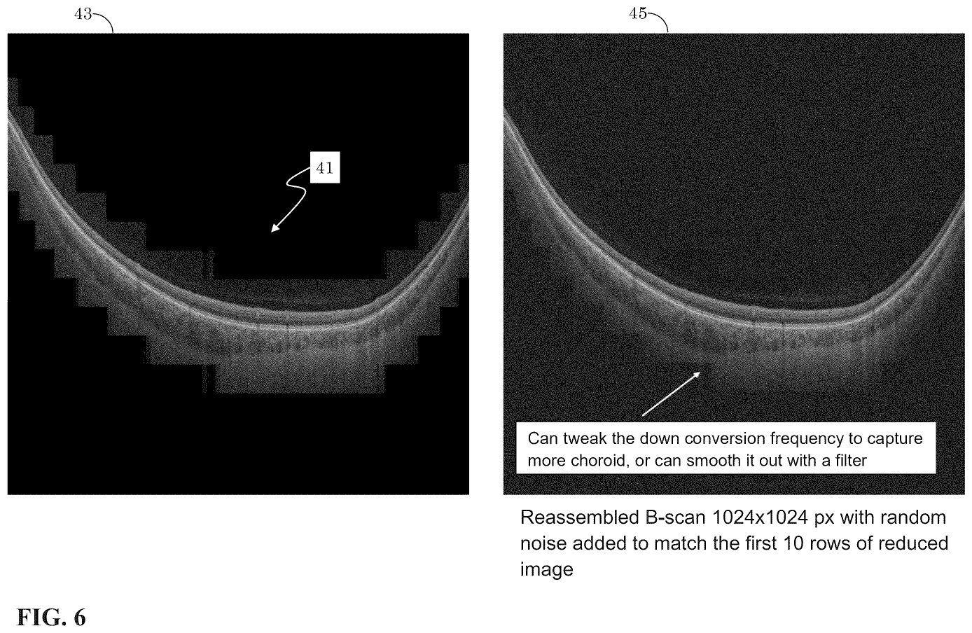

FIG. 6 shows the reassembly of row-shifted A-scan 33 of FIG. 5. In the present example, the row-shifted and decimated A-scans are reassembled into a standard B-scan 41 for clinical evaluation. Note that the image 43 (on the left) shows only the data 41 that is transmitted (the rest of image 43 is black). For visualization purposes, random noise can be added to the black regions of image 43 and image filtering at the edges performed (not shown) to create a more natural looking B-scan 45.

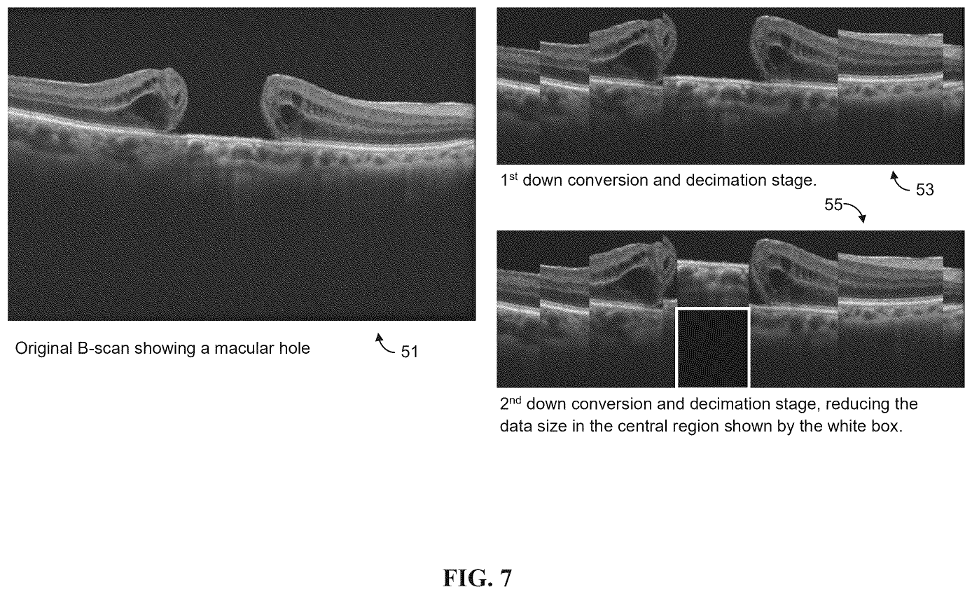

FIG. 7 shows the advantage of a secondary decimation stage. Image 51 shows a retina with the macular hole. In this case, large depth features associated with a macular hole limit the amount of decimation that can be performed in the 1st stage (on the ADC), shown in the top right 53. If the decimation was increased at the ADC, portions of the B-scan would be cropped out of the image and lost. In the middle right, a secondary down conversion and decimation stage 55 further reduces the data size in the central regions.

When transferring the data to the GPU/GPGPU/PC, the FPGA can optionally sort the A-scans according to the total amount of decimation (and samples per A-scan). When sorting the data this way, the original column numbers of each A-scan are also attached to the A-scan to allow for proper B-scan reconstruction.

OCT reconstruction typically uses a fixed number of samples per A-scan, which optimizes the parallel processing time. By sorting the A-scans that are transferred to the PC according to the samples per A-scan, blocks of A-scans can still be reconstructed in parallel. In this case, different configuration parameters are applied for each A-scan sample length used in the reconstruction, and the data sorting is recorded so that the reconstructed A-scan blocks can be inserted into the correct locations of the B-scan for visualization.

### Analog Implementation:

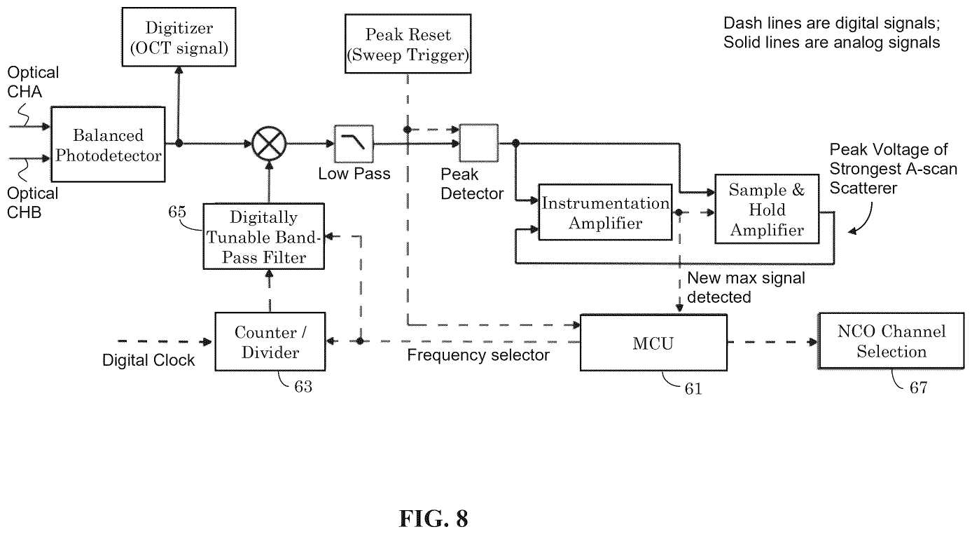

FIG. 8 shows an example of using analog components instead of digitally using an FPGA, as described above. The present embodiment as an adaptation using analog axial tracking. The present approach electronically adjusts the analog down conversion of the OCT signal using a high-speed digital clock that is divided and filtered into an analog signal. The MCU 61 (microcontroller unit) steps through the depth range by digitally adjusting the divider 63 and bandpass filter 65 in discrete counter steps that correspond to the NCO channel spacing. The counter value corresponding to the maximum signal strength of the filtered A-scan signal (corresponding to the retina) is recorded on the microcontroller (MCU) 61. The microcontroller provides a new counter value for each laser sweep. The appropriate NCO channel is then selected (see 67) for digital down conversion, filtering and decimation.

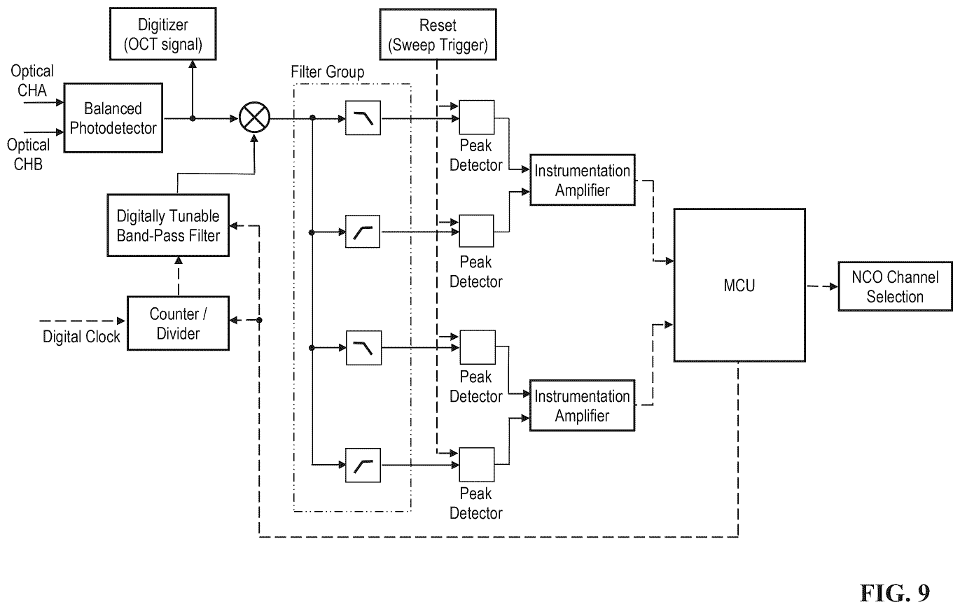

FIG. 9 is a related analog embodiment. Tracking can be implemented by adapting the 4-digital filter embodiment above with analog down conversion and analog filtering and peak detection.

The present, related analog embodiment directs the down converted analog OCT signal to the digitizer and omits digital down conversion on the ADC entirely. This all-analog embodiment that performs tracking and down conversion could save on the cost of the ADC since the sampling rate would be lowered, and digital down conversion would not be a required chip feature. Since the data required for a limited depth range remains the same regardless of whether the first stage down conversion is performed before or after digitization, the data bandwidth and cost would remain the same for the FPGA, GPU, cloud costs, etc. In this embodiment, some additional analog filters would be required after down-conversion to remove unwanted out-of-band harmonics, noise, and other artifacts. One potential downside to sending the down converted analog signal to the digitizer is that better down-conversion and filtering performance may be required as compared to the performance required for axial tracking only. For axial tracking, harmonics that are significantly below the signal strength will have little impact, whereas they may show up as unwanted artifacts in reconstructed images.

Another related embodiment uses the FPGA to generate the digital clock and perform the counter/divider functionality. The resulting frequency clock is filtered using the digitally tunable band pass filter to create the sinusoid to mix with the OCT signal. In this and related embodiments, the digitally tunable band pass filter may be replaced with discrete analog band-pass filters, with the desired signal path selected digitally.

Another embodiment uses at least one voltage-controlled oscillator to generate a sinusoidal waveform at different local oscillator (LO) frequencies. The LO is optionally upconverted to cover the full OCT frequency band with one or more frequency multipliers or mixers and one or more amplifiers.

Another embodiment uses at least one direct digital synthesis (DDS), digital signal processor (DSP), or a digital-to-analog converter (DAC), to generate a sinusoidal waveform at different local oscillator (LO) frequencies. The LO is optionally upconverted to cover the full OCT frequency band with one or more frequency multipliers or mixers and one or more amplifiers.

An alternative to a second filtering, down conversion, and decimation stage would be to acquire OCT data sets by default with decimation that's suitable for typical cases and to rescan patients with a smaller amount of decimation when a larger depth is required. This is a suboptimal solution since it requires rescanning patients after reviewing the reconstructed OCT images and noting that clinically useful data is cropped from the image. These extra steps, and the time required to perform a rescan, may disrupt the clinical workflow of a busy practice.

Another alternative to a second filtering, down conversion, and decimation stage would be the acquire OCT data sets by default with a decimation amount that's suitable for the atypical (large depth) cases and then to remove the sparse unnecessary data from a reconstructed OCT B-scan with post-processing software. This is a suboptimal solution since it adds time to the processing chain prior to writing the data to storage memory, potentially requiring a more expensive and faster GPU.

Hereinafter is provided a description of various suitable hardware and architectures.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

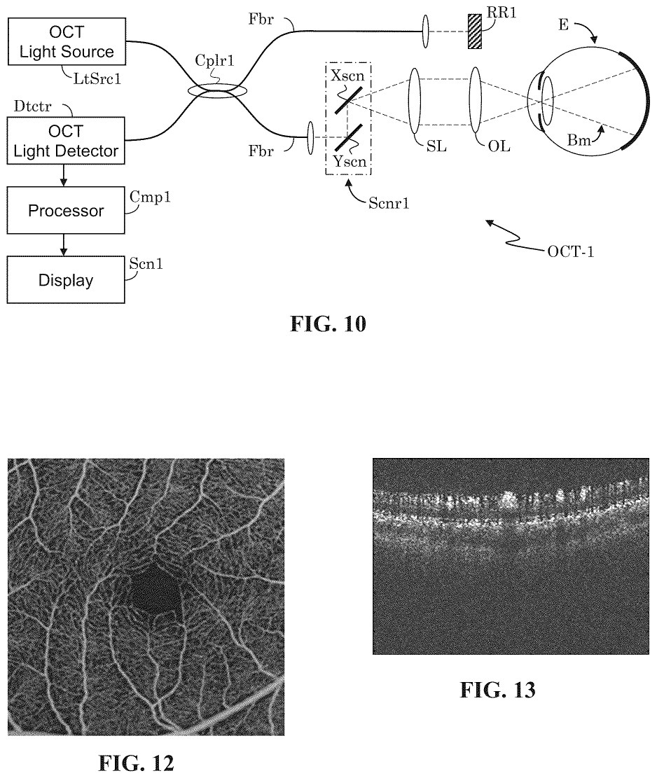

FIG. 10 illustrates a generalized frequency domain optical coherence tomography (FD-OCT) system used to collect 3D image data of the eye suitable for use with the present system. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL (or telecentric scan lens) is used to scan a light beam across the sample. The scan lens helps ensure the light beam's focal point (or focal line) moves linearly and consistently across a flat imaging plane. Together with the ophthalmic lens OL, the light beam is focused onto the sample. The present example illustrates a scan beam that is scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xscn for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner may be used to scan the line-field beam across the sample to span the desired FOV. If the scan beam were a full-field beam (e.g., a full-field OCT), no scanner may be used, and the full-field light beam may be applied across the entire, desired FOV at once.

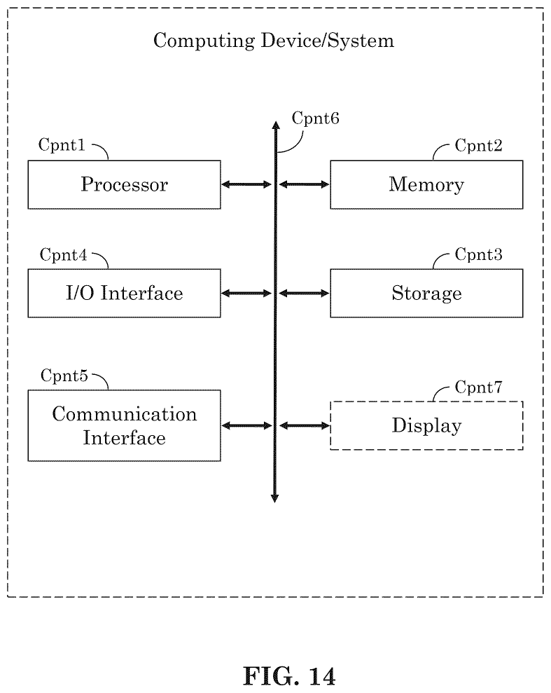

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retro-reflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 14. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13): 2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005)). In time-domain systems, the reference arm has a tunable optical delay to generate interference. Balanced detection systems are typically used in TD-OCT and SS-OCT systems, while spectrometers are used at the detection port for SD-OCT systems. The systems and methods described herein could be applied to any type of OCT system. Various aspects of the disclosure could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

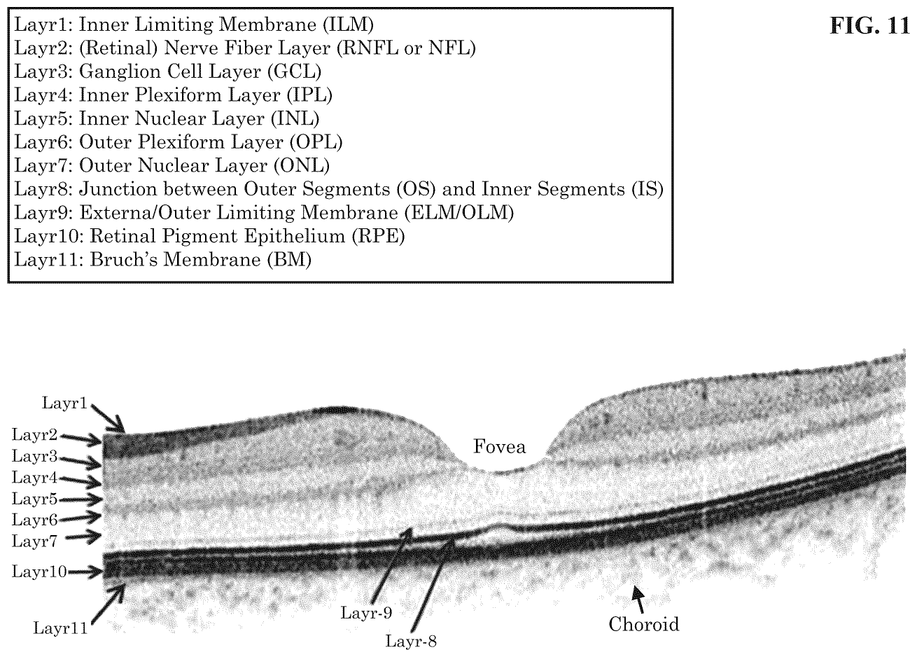

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 11. An OCT B-scan of the retinal provides a view of the structure of retinal tissue. For illustration purposes, FIG. 11 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, all of which are herein incorporated in their entirety by reference). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644 herein incorporated in its entirety by reference). OCT systems that provide an angiography imaging functionality may be termed OCT angiography (OCTA) systems.

FIG. 12 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 13 shows an exemplary B-scan of a vasculature (OCTA) image. As illustrated, structural information may not be well-defined since blood flow may traverse multiple retinal layers making them less defined than in a structural OCT B-scan, as shown in FIG. 11. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique requiring the injection of a dye to observe vascular flow in the retina. Additionally, in dry age-related macular degeneration, OCTA has been used to monitor a general decrease in choriocapillaris flow. Similarly in wet age-related macular degeneration, OCTA can provide a qualitative and quantitative analysis of choroidal neovascular membranes. OCTA has also been used to study vascular occlusions, e.g., evaluation of nonperfused areas and the integrity of superficial and deep plexus.

### Computing Device/System

FIG. 14 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the systems and methods have been described in conjunction with several specific embodiments, it is evident to those skilled in the art that many further alternatives, modifications, and variations will be apparent in light of the foregoing description. Thus, the disclosure is intended to embrace all such alternatives, modifications, applications and variations as may fall within the spirit and scope of the appended claims.

1. An optical coherence tomography system comprising:
a light source for generating a beam of light;
a beam divider for directing a first portion of the light into a reference arm and a second portion of the light into a sample arm;
optics for directing the light in the sample arm to one or more locations on a sample;
a detector for receiving light returning from the sample and reference arms and generating an analog OCT signal in response thereto;
a processor implementing the following steps:
digitizing the OCT signal;
performing a first down conversion and decimation on the digitized OCT signal to isolate a group of OCT signal frequencies of interest within a first low frequency bandwidth;
identifying a bandwidth and a center frequency that characterizes the group of OCT signal frequencies of interest within the first low frequency bandwidth; and
using the bandwidth and center frequency to perform a second down conversion and decimation step to further isolate the group of OCT signal frequencies of interest within a second low frequency bandwidth that is smaller than the first low frequency bandwidth.

2. The system of claim 1, wherein in the first down conversion and decimation step, the digitized OCT signal is separately mixed with a plurality of local oscillators to produce a plurality of intermitted heterodyned signals, and a select intermittent heterodyned signal is identified as the bandwidth that characterizes the group of OCT signal frequencies of interest.

3. The system of claim 2, wherein the select intermittent heterodyned signal is of highest signal strength among the plurality of heterodyned signals.

4. The system of claim 1:
wherein the OCT system is a swept source OCT system;
wherein the OCT signal is resampled using a clock that is derived from the wavenumber k dependence of a sweep; and
wherein a phase correction term is applied to the heterodyned signal based on a local oscillator used in the first down conversion.

5. The system of claim 4, wherein the phase correction term is obtained by resampling the signal phase of the local oscillator with the same time values used to resample the OCT signal.

6. The system of claim 4:
wherein the phase correction term is modeled using an estimate of the local oscillator frequency; and
wherein an error in this estimation is identified as an increase in a point-spread function (PSF) width, and adjust accordingly.

7. A method of reducing the amount of raw data acquired from an OCT system, the method comprising:
performing a first down conversion on an OCT signal from the OCT system to isolate a group of OCT signal frequencies of interest within a first low frequency bandwidth;
filtering and digitizing the first low frequency bandwidth of the OCT signal;
finding a bandwidth and a center frequency that characterizes the group of OCT signal frequencies of interest within the first low frequency bandwidth; and
using the bandwidth and center frequency to perform a second down conversion and decimation step to further isolate the group of OCT signal frequencies of interest within a second low frequency bandwidth that is smaller than the first low frequency bandwidth.
